(19) United States
(12) Patent Application Publication
(10) Pub. No.: US 2012/0130262 A1
(43) Pub. Date: May 24, 2012

(54) Method and Apparatus For Early Warning of Critical Care Patient Hemodynamic Instability

(76) Inventor: Balachandra R. Deshpande, Novi, MI (US)

(21) Appl. No.: 13/076,784

(22) Filed: Mar. 31, 2011

Related U.S. Application Data
(60) Provisional application No. 61/415,915, filed on Nov. 22, 2010.

(51) Int. Cl. A61B 5/026

(52) U.S. Cl. 600/504

(57) ABSTRACT

A method and apparatus for providing a computing environment for a user which gives early warning of critical care patient instability. The method and apparatus use the entropy of monitored channels which are paired, each channel being paired once with each other channel. The entropies within each pair are compared to create an information exchange ratio. The information exchange ratios are integrated and a maximum of the integrated information exchange ratios is determined. Then, an alarm condition occurs at a user determined percentage of the maximum integrated information exchange ratio.

### CROSS-REFERENCE TO RELATED APPLICATIONS

This application claims priority to and the benefit of U.S. Provisional Application 61/415,915 filed Nov. 22, 2010, which is herein incorporated by reference in its entirety.

### FIELD OF THE INVENTION

The present disclosure is in the technical field of data monitoring. More particularly, the present disclosure focuses on data monitoring of critical care patient hemodynamic stability for early warnings.

### BACKGROUND OF THE INVENTION

Intensive Care Unit (ICU) patients require high-intensity monitoring and life support. Patients are admitted to an ICU for a variety of reasons such as respiratory compromise, hemodynamic compromise, myocardial ischemia or infarction, neurological compromise, gastrointestinal issues, renal issues, metabolic issues, postoperative care, and the like.

### BRIEF SUMMARY OF THE INVENTION

The present disclosure discusses a method for evaluating the monitored parameters, also known as channels, of an ICU patient. The monitored parameters generate data which can be compared and evaluated. Examples of monitored parameters include blood pressure, body temperature, blood sugar, and the like.

In the method, data from two parameters is paired and compared. Data from each parameter is paired and compared with each other parameter one time. For example, two parameters would enable one pair, three parameters would enable three pairs, four parameters would enable six pairs, and 40 parameters would enable 780 pairs. There is no theoretical limit to the number of parameters or corresponding pairs.

When pairing and comparing parameters, each parameter has a 0-100% range which is user determined. For example, body temperature may have a linear range of 0-100% which corresponds to 90-110 degrees Fahrenheit. In some cases, a parameter may have a linear range of 0-100% which corresponds to a non-linear parameter which has a logarithmic relationship, exponential relationship, or the like.

Each pair is monitored for the inter-relationship between the two parameters over time. The information exchanged between any pair of channels can be measured by measuring the individual channel entropies and the joint entropy between the pair.

I(X;Y)=H(X)+H(Y)−H(X,Y)

H(X)−entropy of channel X=Σ−p(x)ln(p(x))

H(Y)−entropy of channel Y=Σ−p(y)ln(p(y))

H(X,Y)−Joint entropy=Σ−p(x,y)ln(p(x,y))

Here p(x) refers to the probability of channel X=x, and so on; while p(x,y) refers to the joint probability of channels X=x, Y=y.

By measuring this information exchange between every possible pair of channels, summing the total information exchanged and tracking the level of this summation allows us to distinguish between a true alarm (system failure) and a false alarm. By intelligently selecting the sample size window for computations, we can also detect and provide early warnings for potential system failures.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

The following detailed description of the invention is merely exemplary in nature and is not intended to limit the invention or the application and uses of the invention. Furthermore, there is no intention to be bound by any theory presented in the preceding background of the invention or the following detailed description of the invention.

### BRIEF DESCRIPTION OF THE DRAWINGS

FIG. 1 shows a scatter plot with data used in a sample computation.

FIG. 2 shows the change of two parameters or channels over time.

FIG. 3 shows a relationship between two parameters or channels over time.

FIG. 4 shows a second change of two parameters or channels over time.

FIG. 5 shows a second relationship between two parameters or channels over time.

FIG. 6 shows a third change of two parameters or channels over time.

FIG. 7 shows a third relationship between two parameters or channels over time.

FIG. 8 shows a relationship between two parameters or channels over four consecutive time frames.

FIG. 9 is a block diagram of a typical computing environment used for implementing embodiments of the present disclosure.

### DETAILED DESCRIPTION OF THE INVENTION

In an Intensive Care Unit (ICU), patients are constantly monitored via devices which sample vital data (heart rate, systolic pressure, CO2 concentration, keratinin levels, temperature, etc.) at a certain frequency. The ICU is, by definition, a “multi-disciplinary environment”. A major market segment in patient health monitors relates to this multi-parameter patient monitoring (MPM) technology. MPM includes devices which are used to monitor more than one patient parameter. These monitors track multiple parameters such as temperature, blood pressure, oxygen, respiration etc and are used in ICU's, during surgery and any emergency care. MPM is becoming a part of integrated healthcare information systems as this provides prospects of reducing healthcare costs and enhancing patient safety. Each individual channel is typically set to indicate an unsafe condition when the monitored signal exceeds pre-defined levels. This logic however can lead to multiple false alarms because a single channel can cross its preset levels for a variety of physiologic and non-physiologic reasons. Furthermore, the human body is a highly “coupled” system, which means that issuing alarms based on considering channels independently of one another is not always a good indicator of a real crisis. Thus a major challenge that arises with MPM is to distinguish a true alarm from a false one.

Being able to measure the “overall stability” of the patient—i.e. taking into account the multi-disciplinary character of the data available in an ICU—would be of great value to critical care medicine. Such a measurement would not only quantify the effects of medications and/or the magnitude and severity of crises, it would also help to establish early-warning signals, indicating the onset of a new crisis.

Current best practices include tracking trends in multiple channels and using this to provide early warnings. This requires calculating median values of signals and comparing them to historic trend predictors. However this ignores the fact that there is significant information exchange between the various sub-systems which are being monitored and a loss of information between the sub-systems can lead to overall systemic failure. Furthermore, standard statistical measures of information exchange, such as correlation coefficients fail to work in situations where the data is highly non-linear and non-stationary.

The approach presented here includes measuring the information exchanged between all pairs of monitored channels and using this as an indicator of the relative health of the system (in this case the patient). The concept of information entropy, introduced by Claude Shannon in 1949 serves as the conceptual and theoretical basis for this invention.

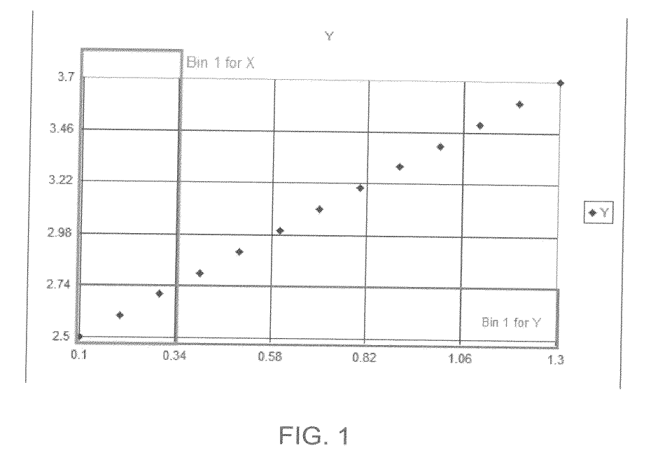

FIG. 1 shows a scatter plot with data used in a sample computation. The computation starts by selecting a pair of parameters, and creating a scatter plot between the pair. For a dataset with p parameters, the total number of available pairings that will be plotted is given by p*(p−1)/2.

Based on the ranges of the parameters selected, each axis will be divided into an odd number of bins or categories. Bins for the horizontal (X) axis are vertical and vice versa. An odd numbered division allows the determination of a central range of values. Typically, for small sample sizes (n<30), 3 bins are made. For a high density dataset (with no missing values) and more than 100 samples, 7 bins are made. For datasets in between, 5 bins are the best choice.

After dividing the X and Y axis into 5 bins each, we are left with 25 cells. The following process steps will be repeated for all pairs of parameters.

Process Step 1—computing Shannon entropy for X and Y variables:

Substep 1: Count the number of data points within each bin for X axis. Let the number of points in each bin be n_{b}.

Substep 2: Compute the probability of finding a sample within this bin as n_{b}/N, where N is the total number of points in the scatter plot.

Substep 3: Compute the natural logarithm: ln(n_{b}/N) for each bin.

Substep 4: Compute the Shannon entropy for the X variable by summing the natural logs calculated above for the 5 bins.

Substep 5: Shannon entropy for variable X, H(X)=−Σ ln(n_{b}/N).

Substep 6: Repeat process substeps 1 through 4 for the Y variable to obtain H(Y)

Process Step 2—computing Joint Entropy:

Substep 1: Count the number of data points within each cell. For a 5 bin discretization, there will be 25 cells. Let the number of points within a cell be given by n_{c}.

Substep 2: Compute the probability of finding a sample within a cell as n_{c}/N.

Substep 3: Compute the natural logarithm: ln(n/N) for each cell.

Substep 4: Compute the Joint entropy for X and Y by summing the natural logs calculated above for all 25 cells.

Substep 5: Joint entropy for variables X and Y, H(X,Y)=−τ ln(n_{c}/N)

Process Step 3—computing Mutual Information, I(X;Y):

Substep 1: Compute Mutual Information, I(X;Y)=H(X)+H(Y)−H(X,Y).

Substep 2: Compute the Information Exchange Ratio (IER), IER=I(X;Y)/H(X,Y).

Repeat these process steps p*(p−1)/2 times to cover all pairs of variables. Add the total IER computed to provide an integrated IER.

The following figures illustrate the concept and how it is incorporated in a device that is capable of integrating multiple data streams to provide early warnings or alerts.

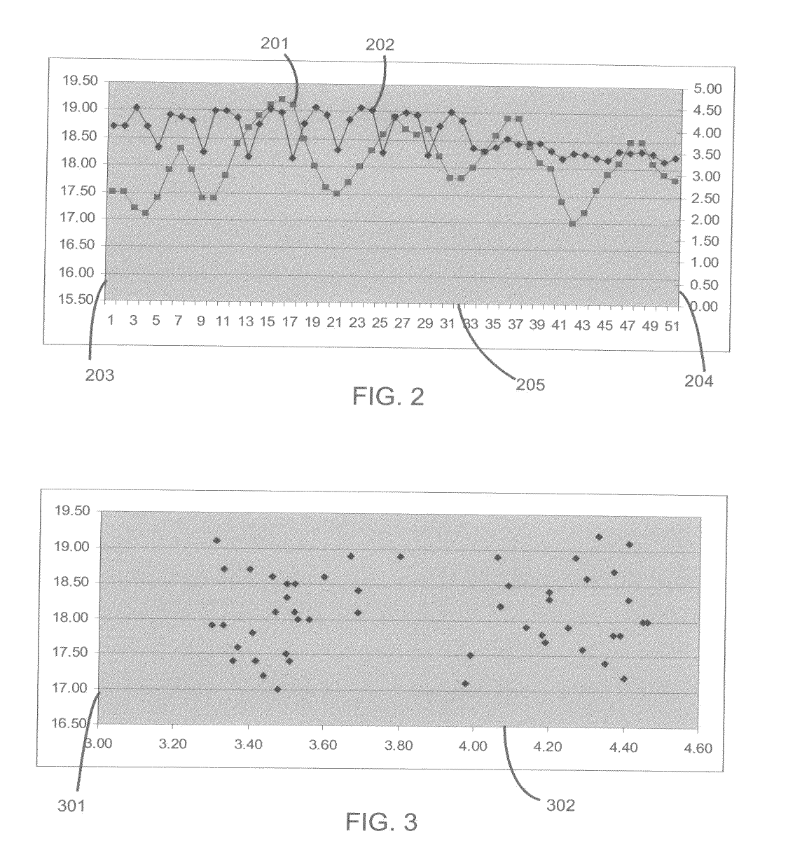

FIG. 2 shows the change of two parameters or channels over time. A first channel 201 and second channel 202 are monitored over time. The first channel 201 corresponds to the left y-axis 203 and the second channel 202 corresponds to the right y-axis 204. The x-axis 205 measures time in 10 second increments.

FIG. 3 shows a relationship between two parameters or channels over time. FIG. 2 and FIG. 3 use the same raw data. The relationship is shown as a scatter chart. The y-axis 301 corresponds to left y-axis 203. The x-axis 302 corresponds to right y-axis 204. There seems to be a very weak or non-existent relationship between the two channels. This knowledge can be effectively captured using I(X;Y) which in this case is 0.16. However, it is convenient to express this as a fraction of the joint information entropy, by dividing the I(X;Y) by H(X,Y). The value in this case is 6%, we shall call this the information exchange ratio.

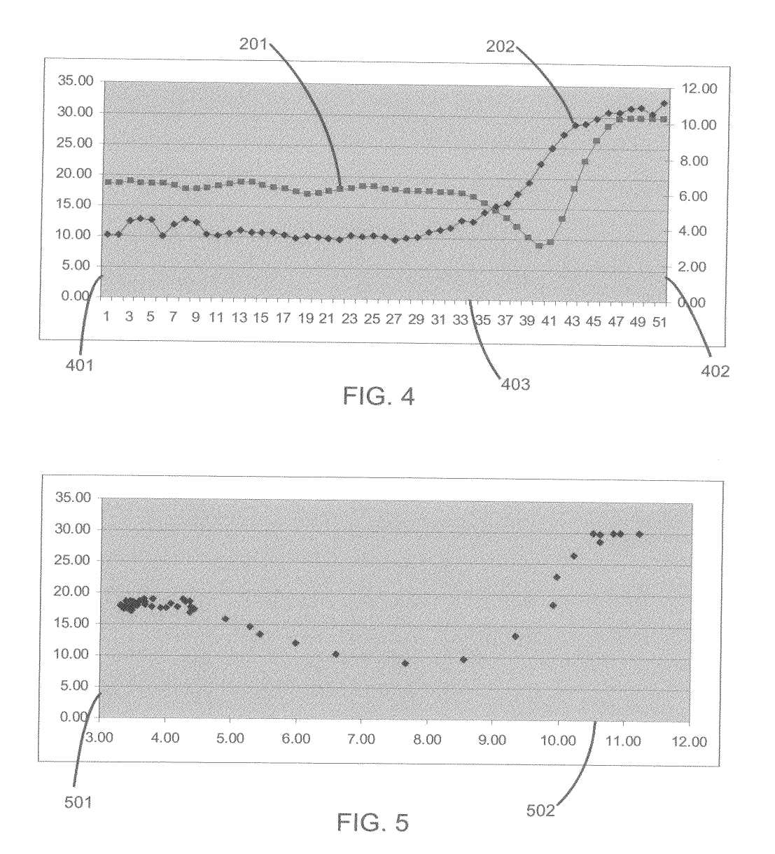

FIG. 4 shows a second change of two parameters or channels over time. A first channel 201 and second channel 202 are monitored over time and are the same channels being monitored in FIG. 2. The first channel 201 corresponds to the left y-axis 401 and the second channel 202 corresponds to the right y-axis 402. The x-axis 403 measures time in 10 second increments. The two parameters suddenly exhibit a distinct pattern of inter-relationship. As one channel increases in value, so does the other—at least toward the end of the monitored time window.

FIG. 5 shows a second relationship between two parameters or channels over time. FIG. 4 and FIG. 5 use the same raw data. The relationship is shown as a scatter chart. The y-axis 501 corresponds to left y-axis 401. The x-axis 502 corresponds to right y-axis 402. The corresponding information exchange is now 0.82 and the information exchange ratio is 68%.

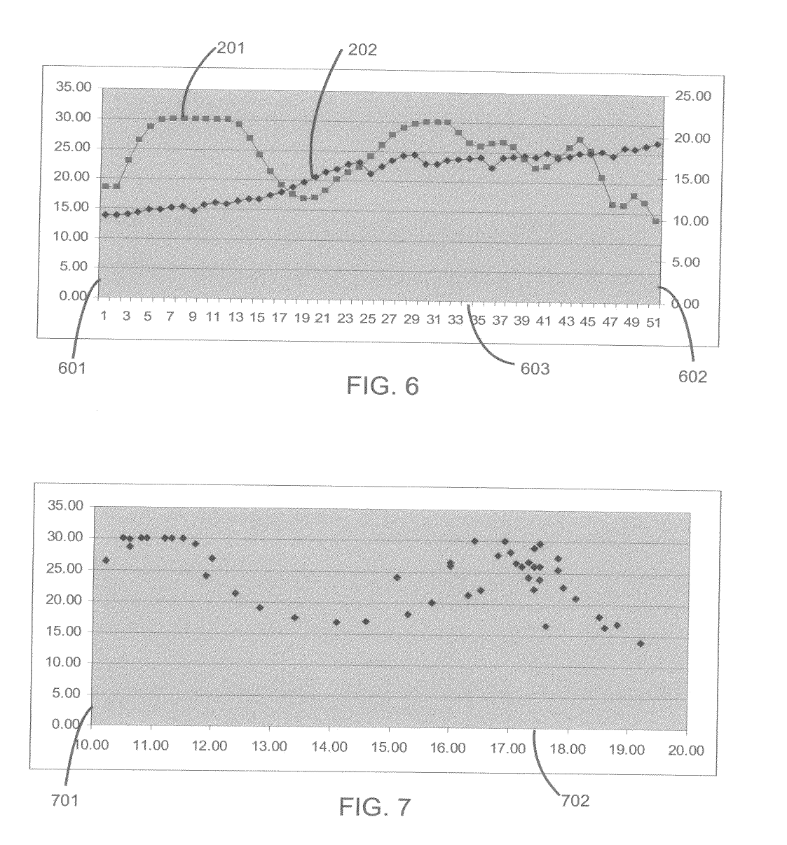

FIG. 6 shows a third change of two parameters or channels over time. A first channel 201 and second channel 202 are monitored over time and are the same channels being monitored in FIG. 2. The first channel 201 corresponds to the left y-axis 601 and the second channel 202 corresponds to the right y-axis 602. The x-axis 603 measures time in 10 second increments. We see that the trend between the two channels which was quite evident in FIG. 4 and FIG. 5 is now slowly “dissolving” and the corresponding information exchange ratio is 0.28—only slightly higher than in FIG. 2 and FIG. 3.

FIG. 7 shows a third relationship between two parameters or channels over time. FIG. 6 and FIG. 7 use the same raw data. The relationship is shown as a scatter chart. The y-axis 701 corresponds to left y-axis 601. The x-axis 702 corresponds to right y-axis 602. The information exchange ratio is 11%, which is significantly lower than FIG. 4 and FIG. 5, and marginally higher than FIG. 2 and FIG. 3.

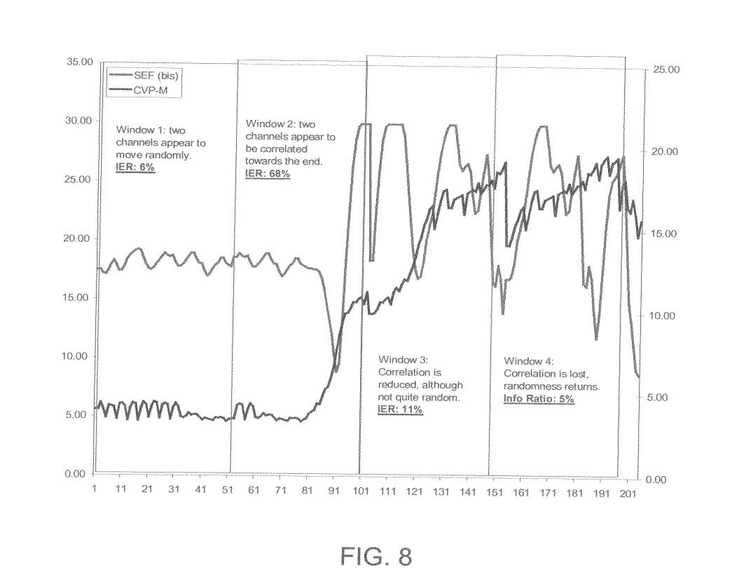

FIG. 8 shows a relationship between two parameters or channels over four consecutive time frames. This image shows the sequence of channel dynamics: in the first window 801 (or step 1), the two channels are more or less random with respect to each other. This however changes at the end of second window 802 (step 2) where they appear to increase in tandem or being correlated to each other. The correlation weakens significantly in the third window 803 window 3 (step 3) and finally reverts back to randomness in the fourth window 804.

The algorithm monitors this type of conjoint behavior between all possible pairs of channels (for example a typical ICU with 40 channels would have 780 combinations to be monitored dynamically). The information exchange ratio when integrated across all combinations of channels shows a strong correlation with the overall structural stability of the system, in particular the hemodynamic instability of the patient in an ICU.

In our studies with animal and human data, we have identified that any significant reduction in the information exchange ratio, once it attains a high nominal value, is strongly correlated with hemodynamic instability. In particular, when this ratio drops more than 30% from a previously attained peak value, it signals a hemodynamic instability.

Our device performs the following major tasks automatically:

After an initialization period, it integrates all the information exchanged between channel pairs at periodic monitoring intervals: 1) The frequency of computation can be adjusted by the user based on patient condition. For patients with higher criticality, this interval can be made equal to the most frequent data collection period of any of the available channels; 2) If the user does not select a monitoring interval, the device automatically sets the interval to the one which gives the least amount of period to period information exchange ratio fluctuation during the initialization phase.

If the information exchange ratio monotonically decreases by more than 30% from a previous peak value, the device signals an instability alert.

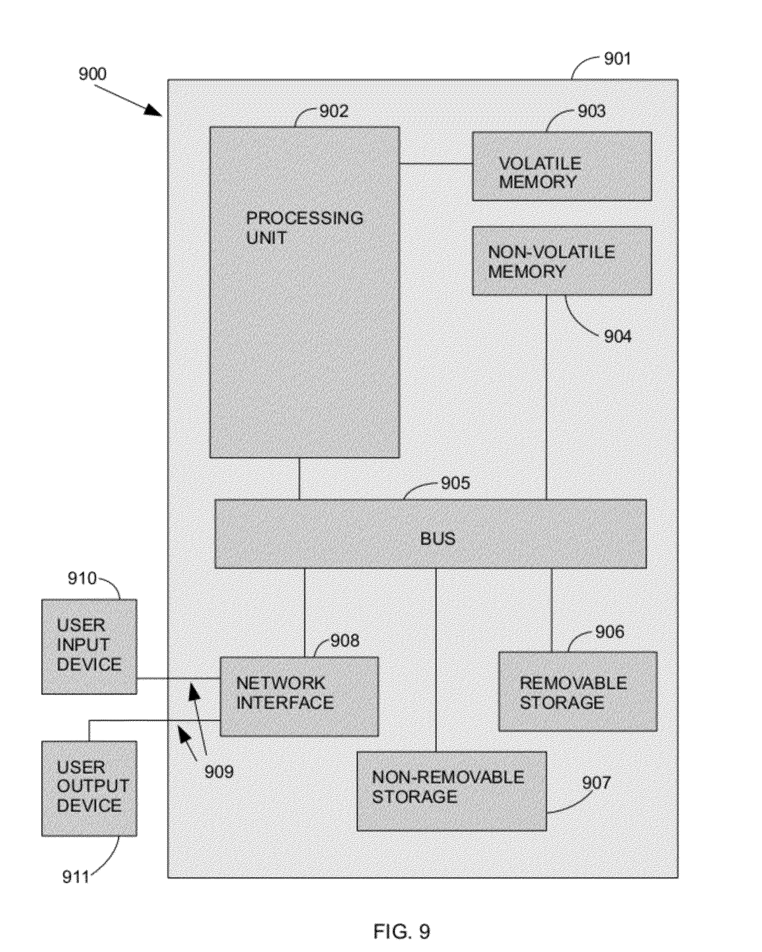

FIG. 9 is a block diagram of a typical computing environment used for implementing embodiments of the present disclosure. FIG. 9 and the following discussion are intended to provide a brief, general description of a suitable computing environment in which certain embodiments of the present disclosure may be implemented.

FIG. 9 shows a computing environment 900, which can include but is not limited to, a housing 901, processing unit 902, volatile memory 903, non-volatile memory 904, a bus 905, removable storage 906, non-removable storage 907, a network interface 908, ports 909, a user input device 910, and a user output device 911.

Various embodiments of the present subject matter can be implemented in software, which may be run in any suitable computing environment. The embodiments of the present subject matter are operable in a number of general-purpose or special-purpose computing environments. Some computing environments include personal computers, server computers, hand-held devices (including, but not limited to, telephones and personal digital assistants (PDAs) of all types), laptop devices, multi-processors, microprocessors, set-top boxes, programmable consumer electronics, network computers, minicomputers, mainframe computers, distributed computing environments, analyzers designed to read multiple inputs from a critical care patient, and the like to execute code stored on a computer readable medium. The embodiments of the present subject matter may be implemented in part or in whole as machine-executable instructions, such as program modules that are executed by a computer. Generally, program modules include routines, programs, objects, components, data structures, and the like to perform particular tasks or to implement particular abstract data types. In a distributed computing environment, program modules may be located in local or remote storage devices.

A general computing device, in the form of a computer, may include a processor, memory, removable storage, non-removable storage, bus, and a network interface.

A computer may include or have access to a computing environment that includes one or more user input modules, one or more user output modules, and one or more communication connections such as a network interface card or a USB connection. The one or more output devices can be a display device of a computer, computer monitor, TV screen, plasma display, LCD display, display on a digitizer, display on an electronic tablet, and the like. The computer may operate in a networked environment using the communication connection to connect one or more remote computers. A remote computer may include a personal computer, server, router, network PC, a peer device or other network node, and/or the like. The communication connection may include a Local Area Network (LAN), a Wide Area Network (WAN), and/or other networks.

Memory may include volatile memory and non-volatile memory. A variety of computer-readable media may be stored in and accessed from the memory elements of a computer, such as volatile memory and non-volatile memory, removable storage and non-removable storage. Computer memory elements can include any suitable memory device(s) for storing data and machine-readable instructions, such as read only memory (ROM), random access memory (RAM), erasable programmable read only memory (EPROM), electrically erasable programmable read only memory (EEPROM), hard drive, removable media drive for handling compact disks (CDs), digital video disks (DVDs), diskettes, magnetic tape cartridges, memory cards, memory sticks, and the like. Memory elements may also include chemical storage, biological storage, and other types of data storage.

“Processor” or “processing unit” as used herein, means any type of computational circuit, such as, but not limited to, a microprocessor, a microcontroller, a complex instruction set computing (CISC) microprocessor, a reduced instruction set computing (RISC) microprocessor, a very long instruction word (VLIW) microprocessor, an explicitly parallel instruction computing (EPIC) microprocessor, a graphics processor, a digital signal processor, or any other type of processor or processing circuit. The term also includes embedded controllers, such as generic or programmable logic devices or arrays, application specific integrated circuits, single-chip computers, smart cards, and the like.

Embodiments of the present subject matter may be implemented in conjunction with program modules, including functions, procedures, data structures, application programs, etc. for performing tasks, or defining abstract data types or low-level hardware contexts.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

I claim:

1. A method of providing a computing environment for a user which gives early warning of critical care patient instability, comprising:
measuring a plurality of critical care patient channels via the computing environment periodically to determine an entropy corresponding to each channel;
comparing each channel entropy to each other channel entropy to create corresponding channel pairs;
assessing the two entropies within each channel pair to determine an information exchange ratio for each channel pair;
integrating the information exchange ratios of all channel pairs;
measuring the integrated information exchange ratios of the channel pairs over time to determine a maximum value which corresponds to maximum critical care patient stability; and
creating an alarm condition so that the computing environment notifies the user of critical care patient instability when the value of the integrated information exchange ratios of the channel pairs falls below a user determined percentage of the maximum value of the integrated information exchange ratios of the channel pairs.

2. The method of claim 1, wherein the user determined percentage is 70%.

3. The method of claim 1, wherein the periodic measurement of the plurality of critical care patient channels occurs with a frequency equal to the most frequent data collection period available to any of the channels.

4. The method of claim 1, wherein the periodic measurement of the plurality of critical care patient channels occurs with a frequency equal to the least frequent data collection period available to any of the channels.

5. The method of claim 1, wherein the periodic measurement of the plurality of critical care patient channels occurs with a user determined frequency.

6. The method of claim 1, further comprising assigning an index value to the integrated information exchange ratios of the channel pairs.

7. An apparatus that incorporates a computing environment for a user which gives early warning of critical care patient instability, comprising:
measuring a plurality of critical care patient channels via the computing environment periodically to determine an entropy corresponding to each channel;
comparing each channel entropy to each other channel entropy to create corresponding channel pairs;
assessing the two entropies within each channel pair to determine an information exchange ratio for each channel pair;
integrating the information exchange ratios of all channel pairs;
measuring the integrated information exchange ratios of the channel pairs over time to determine a maximum value which corresponds to maximum critical care patient stability; and
creating an alarm condition so that the computing environment notifies the user of critical care patient instability when the value of the integrated information exchange ratios of the channel pairs falls below a user determined percentage of the maximum value of the integrated information exchange ratios of the channel pairs.

8. The apparatus of claim 7, wherein the user determined percentage is 70%.

9. The apparatus of claim 7, wherein the periodic measurement of the plurality of critical care patient channels occurs with a frequency equal to the most frequent data collection period available to any of the channels.

10. The apparatus of claim 7, wherein the periodic measurement of the plurality of critical care patient channels occurs with a frequency equal to the least frequent data collection period available to any of the channels.

11. The apparatus of claim 7, wherein the periodic measurement of the plurality of critical care patient channels occurs with a user determined frequency.

12. The apparatus of claim 7, further comprising assigning an index value to the integrated information exchange ratios of the channel pairs.
